(19) United States
(12) Patent Application Publication
(10) Pub. No.: US 2022/0188934 A1
(43) Pub. Date: Jun. 16, 2022

(54) SYSTEMS AND METHODS FOR GENERATING USER-SPECIFIC WELL-BEING TASKS

(71) Applicant: STATE FARM MUTUAL AUTOMOBILE INSURANCE COMPANY, Bloomington, IL (US)

(72) Inventors: Edward W. Breitweiser, Bloomington, IL (US); Jennifer L. Crawford, Normal, IL (US); Brian Steigerwald, Bloomington, IL (US); Danielle Malan, Downs, IL (US)

(21) Appl. No.: 17/550,475

(22) Filed: Dec. 14, 2021

Related U.S. Application Data
(60) Provisional application No. 63/125,465, filed on Dec. 15, 2020.

(51) Int. Cl. G06Q 40/06; G16H 40/63; G06Q 50/00

(52) U.S. Cl. CPC G06Q 40/06; G06Q 50/01; G16H 40/63

(57) ABSTRACT

Systems and methods for generating user-specific well-being recommendations relating to user interaction with a well-being application are disclosed. User data is obtained via the well-being application and from additional data sources, and such user data is processed to determine user conditions that may serve as triggering events (e.g., major life events or changes in user well-being). When a triggering event occurs, well-being tasks are determined and personalized into a user-specific well-being recommendation to the user of the well-being application. In some embodiments, additional prompts may be used to obtain additional user data to further personalize the user-specific well-being recommendation, which may include monitors user responses to offers for additional information in the well-being application. User personalization may include determining a best time or manner of presenting the recommendation to the user to increase the likelihood of user performance of the recommended well-being tasks.

### CROSS-REFERENCE TO RELATED APPLICATIONS

This application claims the benefit of U.S. Provisional Application Ser. No. 63/125,465 filed Dec. 15, 2020, which is incorporated herein by reference.

### TECHNICAL FIELD

The present disclosure generally relates to systems and methods for monitoring user well-being metrics, and generating user-specific recommendations to improve user well-being.

### BACKGROUND

Electronic wellness devices and software applications provide users a variety of biometric data regarding aspects of their physical well-being. For example, some wearable devices monitor user steps, heart rate, and sleep quality. Although this information is useful for tracking progress toward specific user-defined goals and for automatically generating generic recommendations based on user biometric data, existing techniques do not provide users with tailored recommendations or instructions for other aspects of well-being beyond physical health (e.g., mental, financial, social, or community well-being).

These other aspects of well-being are important to individuals and can interact with physical health, but user-specific data generation and evaluation have presented a significant challenge. Specifically, many of the metrics and recommendations for user actions relating to other types of well-being are impacted by life events and other changes in conditions that are not easily observable by existing methods. Therefore, improved techniques for monitoring user well-being, and generating user-specific recommendations to improve well-being beyond physical well-being are needed.

### SUMMARY

The present embodiments relate to generation of user-specific well-being recommendations relating to well-being tasks to be performed by a user. Such well-being recommendations may be related to mental, financial, social, or community well-being of a user.

In one aspect, a computer-implemented method for generating user-specific well-being recommendations may include, via one or more local or remote processors, servers, transceivers, and/or sensors: (1) monitoring user interactions with a well-being application via a user interface of a client computing device associated with the user; (2) collecting user external data from one or more external data sources; (3) identifying the occurrence of a triggering event associated with a user condition based upon the user interactions and the user external data; (4) determining a well-being recommendation to the user to complete a well-being task based upon (i) the triggering event, and (ii) at least one of the user interactions or the user external data; (5) generating a user-specific recommendation to perform the well-being task; and/or (6) causing the user-specific recommendation to be presented to the user via the user interface of the client computing device. The method may include additional, fewer, or alternate actions, including those discussed elsewhere herein.

For instance, the one or more external data sources may include a social media feed associated with the user, in which case identifying the triggering event may comprise identifying a major life event of the user based upon social media data included in the social media feed. The user condition may include one or more of the following major life events associated with the user or with another person associated with the user: a birth, a death, an adoption, a marriage, a divorce, a change in employment, a retirement, a medical diagnosis, a purchase of real property, a change of residence, or reaching a threshold age.

The well-being task may be a task related to user financial, mental, or social well-being of the user. In some embodiments, the well-being task comprise one or more of the following: collecting financial documents, uploading financial documents to a server associated with the well-being application, participating in a community volunteering activity, connecting with an acquaintance, planning a vacation, reviewing retirement financial plans, performing property maintenance, filing official form documents, updating insurance policy beneficiaries, or providing information to insurance policy beneficiaries.

In some embodiments, the well-being recommendation may be determined in part based upon an indication of the user response to a prompt. In some such embodiments, the computer-implemented method may include (a) determining one or more well-being prompts to the user based upon the triggering event; (b) causing the one or more well-being prompts to be presented to the user via the user interface of the client computing device; and/or (c) receiving an indication of a user response to at least one of the one or more well-being prompts. In further such embodiments, the one or more well-being prompts may include an offer of additional information relating to the user condition, and the user response may comprise accepting the offer of additional information.

In further embodiments, determining the user-specific recommendation may comprise generating an input data set from the user interactions and the user external data relating to the triggering event, applying an artificial intelligence model associated with the triggering event to the input data set to generate a plurality of rating scores associated with respective well-being tasks, and selecting one or more of the well-being tasks to include in the well-being recommendation based upon the rating scores.

Generating the user-specific recommendation may include identifying specific steps to perform the well-being task, identifying specific documents, and/or identifying specific individuals or organizations to contact. In some embodiments, the user-specific recommendation may include an incentive for performing the well-being task within a specified time interval. In some such embodiments, the computer-implemented method may further include: collecting additional data associated with the user following presentation of the user-specific recommendation during the specified time interval; determining completion of the well-being task by the user; and/or causing the incentive to be implemented for the user based upon the completion of the well-being task.

Systems or computer-readable media storing instructions for implementing all or part of the methods described above may also be provided in some aspects. Systems for implementing such methods may include one or more of the following: a client computing device associated with a user of a well-being application, a server associated with a well-being application, one or more additional data sources, one or more communication modules configured to communicate wirelessly via radio links, radio frequency links, and/or wireless communication channels, and/or one or more program memories coupled to one or more processors of any such computing devices or servers. Such program memories may store instructions to cause the one or more processors to implement part or all of the method described above. Additional, fewer, or alternative features described herein below may be included in some aspects.

### BRIEF DESCRIPTION OF DRAWINGS

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

The Figures described below depict various aspects of the applications, methods, and systems disclosed herein. It should be understood that each Figure depicts an embodiment of a particular aspect of the disclosed applications, systems and methods, and that each of the Figures is intended to accord with one or more possible embodiments thereof. Furthermore, wherever possible, the following description refers to the reference numerals included in the following Figures, in which features depicted in multiple Figures are designated with consistent reference numerals.

FIG. 1 illustrates a block diagram of an exemplary well-being application system for monitoring and improving user well-being.

FIG. 2 illustrates a flow diagram of an exemplary user-specific well-being recommendation generation method.

FIG. 3 illustrates a flow diagram of an exemplary data collection method using prompts to obtain additional user data.

FIG. 4 illustrates a flow diagram of an exemplary model training method for training an artificial intelligence model for determining well-being tasks.

FIG. 5 illustrates a flow diagram of an exemplary incentive selection and implementation method for incentives associated with user performance of well-being tasks.

The Figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION

To improve the effectiveness of well-being recommendations to users of a well-being application, the techniques disclosed herein may be used to collect and analyze user data to determine well-being tasks to recommend to users. The recommendations may be further personalized for the users by generating user-specific, well-being recommendations based upon the user data (and, in some instances, using additional user data obtained by prompting the user to respond to prompts). In some embodiments, artificial intelligence models may be trained and applied to the collected data to determine the well-being recommendations for users. In further embodiments, incentives may be determined to further encourage user performance of the recommended well-being tasks, and user performance may be monitored to determine when to implement the incentives. Additional or alternative aspects are disclosed with respect to exemplary embodiments herein.

### EXEMPLARY WELL-BEING APPLICATION SYSTEM

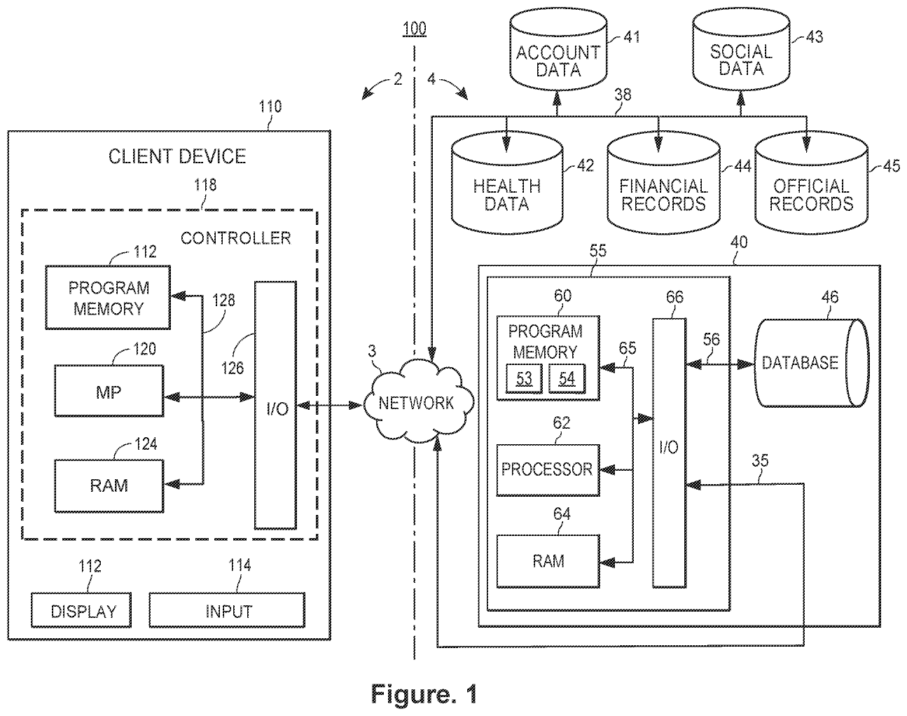

FIG. 1 illustrates a block diagram of an exemplary well-being application system 100 on which the exemplary computer-based methods described herein may be implemented to monitor and improve user well-being. The high-level architecture may include both hardware and software applications, as well as various data communications channels for communicating data between the various hardware and software components.

The well-being application system 100 may be roughly divided into front-end components 2 and back-end components 4. The front-end components 2 may be associated with users of a well-being application to monitor, manage, and/or enhance their well-being related to physical health, mental health, financial condition, document organization, social connection, community involvement, or other aspects of well-being. The back-end components 4 may include hardware and software components implementing aspects of such well-being applications, as well as internal or external data sources associated with user well-being data.

In some embodiments of the system 100, the front-end components 2 may communicate with the back-end components 4 via a network 3. One or more client devices 110 associated with users of the well-being application may communicate with the back-end components 4 via the network 3 to receive data from, and provide data to, back-end components 4 associated with the well-being application. The back-end components 4 may use one or more servers 40 to receive data and data requests from the front-end components 2; process and store received data; access additional data sources; analyze user well-being; provide data to the front-end components 2; and/or perform additional well-being application functions as described herein. The one or more servers 40 may also communicate with other back-end components 4, such as additional data sources 41-45. Some embodiments may include fewer, additional, or alternative components.

The front-end components 2 may be disposed within one or more client devices 110, which may include a desktop computer, notebook computer, netbook computer, tablet computer, or mobile device (e.g., a cellular telephone, smart phone, wearable computer, smart speaker, smart appliance, IoT device, etc.). The client device 110 may include a display 112, an input 114, and a controller 118. In some embodiments, the client device 110 may further include a Global Positioning System (GPS) unit (not shown) to determine a geographical location of the client device 110.

The input 114 may include a “soft” keyboard that is displayed on the display 112 of the client device 110, an external hardware keyboard communicating via a wired or a wireless connection (e.g., a Bluetooth keyboard), an external mouse, or any other suitable user-input device. The input 114 may further include a microphone, camera, or other input device capable of receiving information. The controller 118 includes one or more microcontrollers or microprocessors (MP) 120, a program memory 122, a RAM 124, and an I/O circuit 126, all of which may be interconnected via an address/data bus 128. The program memory 122 may include an operating system, a data storage, a plurality of software applications, and/or a plurality of software routines.

The program memory 122 may include software applications, routines, or scripts for implementing communications between the client device 110 and the server 40 or additional data sources 41-45 via the network 3. For example, the program memory 122 may include a web browser program or application, thereby enabling the user to access web sites via the network 3. As another example, the program memory 122 may include a social media application that receives data from and sends data to a social data source 43 via the network 3. The program memory 122 may further store computer-readable instructions for a program or application associated with one or more well-being applications.

In some embodiments, the controller 118 may also include, or otherwise be communicatively connected to, other data storage mechanisms (e.g., one or more hard disk drives, optical storage drives, solid state storage devices, etc.) that reside within the client device 110. It should be appreciated that although FIG. 1 depicts only one microprocessor 120, the controller 118 may include multiple microprocessors 120. Similarly, the memory of the controller 118 may include multiple program memories 122 or multiple RAMs 124. Although the FIG. 1 depicts the I/O circuit 126 as a single block, the I/O circuit 126 may include a number of different types of I/O circuits. The controller 118 may implement the program memories 122 or the RAMs 124 as semiconductor memories, magnetically readable memories, or optically readable memories, for example.

The various computing devices of the front-end components 2 may communicate with the back-end components 4 via wired or wireless connections to the network 3. The network 3 may be a proprietary network, a secure public internet, a virtual private network or some other type of network, such as dedicated access lines, plain ordinary telephone lines, satellite links, cellular data networks, combinations of these. The network 3 may include one or more radio frequency communication links, such as wireless communication links with client devices 110. The network 3 may also include other wired or wireless communication links with other client devices 110 or other computing devices. Where the network 3 may include the Internet, and data communications may take place over the network 3 via an Internet communication protocol.

The back-end components 4 may include one or more servers 40 configured to implement part or all of the processes related to the well-being application described herein. Each server 40 may include one or more computer processors adapted and configured to execute various software applications and components of the well-being application system 100, in addition to other software applications. The server 40 may further include a database 46, which may be adapted to store data related to user well-being for a plurality of users and/or data relating to recommendations or incentives for users. Such data may include data related to user preferences, user conditions, user policies or accounts, user property, user actions, user incentives, user goals, goal progress, user biometric data, or other well-being data relating to a user, as discussed elsewhere herein, part or all of which data may be collected by or uploaded to the server 40 via the network 3. The server 40 may access data stored in the database 46 or external data sources when executing various functions and tasks associated with the methods discussed elsewhere herein.

The server 40 may have a controller 55 that is operatively connected to the database 46 via a link 56. It should be noted that, while not shown, additional databases may be linked to the controller 55 in a known manner. For example, separate databases may be used for various types of information, such as user profiles, user activity data, or well-being data models. Additional data sources 41-45 may be communicatively connected to the server 40 via the network 3, such as databases maintained by third parties or databases associated with other servers 40. The controller 55 may include a program memory 60, a processor 62 (which may be called a microcontroller or a microprocessor), a random-access memory (RAM) 64, and an input/output (I/O) circuit 66, all of which may be interconnected via an address/data bus 65.

It should be appreciated that although only one processor 62 is shown, the controller 55 may include multiple processors 62. Similarly, the memory of the controller 55 may include multiple RAMs 64 and multiple program memories 60. Although the I/O circuit 66 is shown as a single block, it should be appreciated that the I/O circuit 66 may include a number of different types of I/O circuits. The RAM 64 and program memories 60 may be implemented as semiconductor memories, magnetically readable memories, or optically readable memories, for example. The controller 55 may also be operatively connected to the network 3 via a link 35.

The server 40 may further include a number of software applications stored in a program memory 60. The various software applications on the server 40 may include one or more software applications for monitoring, storing, evaluating, generating, and tracking user well-being data or recommendations. Such software applications may include a well-being evaluation module 53 configured to generate user well-being metrics or identify user well-being conditions, as well as artificial intelligence (AI) models 54 trained and used for generating user well-being recommendations, as discussed further below. The various software applications may be executed on the same computer processor or on different computer processors.

The back-end components 4 may further include one or more additional data sources 41-45 providing information relating to aspects of user well-being. These additional data sources 41-45 may be configured to communicate with the server 40 through the network 3 via a link 38. The additional data sources 41-45 may include an account data source 41, a health data source 42, a social data source 43, a financial records data source 44, and/or an official records data source 45. Information regarding various aspects of users' physical, mental, social, or financial health may be stored in databases associated with the various additional data sources 41-45, which data may be accessed as part of the methods described herein. In some embodiments, additional or alternative data sources may be accessed to obtain further information relevant to user well-being.

The account data source 41 may maintain user account data for a plurality of user accounts associated with users of client devices 110. In some embodiments, such user account data may include user profiles for such users, which may be associated with various services, such as telecommunications services, financial services, insurance policies, or well-being services. For example, user account data may include information regarding insurance policies, bank accounts, and investment accounts associated with a user.

The health data source 42 may maintain user health data associated with a plurality of users, such as biometrics data from wearable computing devices or electronic medical records. Such user health data may be used to monitor aspects of user physical and mental health. In some embodiments, the user health data may relate to individuals associated with a user of a well-being application, such as young children or elderly parents of the user.

The social data source 43 may maintain user social data associated with a plurality of users, such as users of social media platforms. Such social data may be used to identify life events based upon user profile information updates, user posts, or posts referencing a user. In some embodiments, user posts or metadata regarding user posts may also be analyzed to determine user social connections or mental well-being (e.g., stress levels, connectedness, depression, loss, etc.), which may include performing user sentiment analysis.

The financial records data source 44 may maintain user financial records, such as banking or investment records. In some embodiments, financial records may include credit-related records maintained by credit rating agencies, such as revolving accounts, loans, assets, or contractual agreements (e.g., leases, utility, or other services). Such financial records may be used to determine user financial well-being or to generate recommendations regarding improvements in the user's current financial condition or planning for future events (e.g., collecting, organizing, or reviewing financial documents).

The official records data source 45 may maintain official records regarding a user, such as records maintained by various governmental agencies. Such official records may include user property records, licensure records, birth/death records, benefits records, or other official records associated with a user's well-being. In some embodiments, official records may include notices published in newspapers of records or other reliable non-governmental sources.

Although the well-being application system 100 is shown to include one or a limited number of the various front-end components 2 and of the back-end components 4, it should be understood that different numbers of any or each of these components may be utilized in various embodiments. Furthermore, the database storage or processing performed by the one or more servers 40 and/or additional data sources 41-45 may be distributed among a plurality of components in an arrangement known as “cloud computing.” This configuration may provide various advantages, such as enabling near real-time uploads and downloads of information, as well as providing additional computing resources needed to handle the monitoring, modeling, evaluation, and/or recommendation tasks described herein. This may in turn support a thin-client embodiment of some computing devices of the front-end components 2, such as some client devices 110.

### EXEMPLARY USER-SPECIFIC WELL-BEING RECOMMENDATION GENERATION METHODS

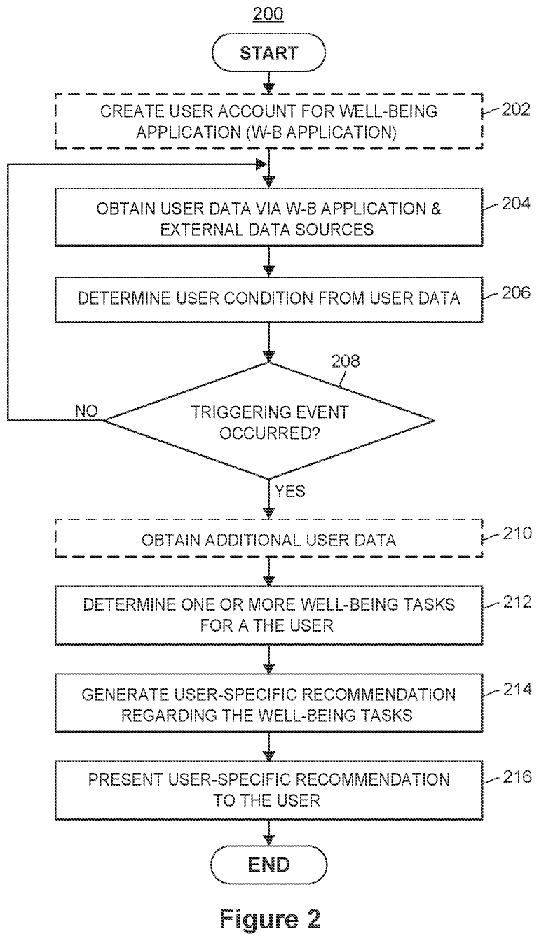

FIG. 2 illustrates a flow diagram of an exemplary user-specific well-being recommendation generation method 200 implemented by the components of the well-being application system 100. The computer-implemented method 200 may be performed periodically or on an ongoing basis to provide users with well-being recommendations tailored to current user conditions. Various parts of the method 200 may be implemented by or performed using the various front-end components 2 and back-end components 4, which may communicate via the network 3, as described above. In some embodiments, a server 40 may perform the method 200 by collecting and processing user data obtained from a well-being application on a client device 110 and from additional data sources 41-45.

The user-specific well-being recommendation generation method 200 begins, in some embodiments, with the creation of a user account for a well-being application associated with the well-being application system 100 (block 202). User data may then be obtained from the client device 110 and additional data sources 41-45 (block 204), and analyzed to determine a user condition (block 206) until occurrence of a triggering event is detected (block 208). In some embodiments, additional user data may be obtained in response to the triggering event (block 210). Following detection of the triggering event, one or more well-being tasks to recommend to the user are determined (block 212) and used to generate a user-specific well-being recommendation (block 214). Such user-specific well-being recommendation is then presented to the user via the client device 110. The method 200 is exemplary only, and other methods may include additional, fewer, or alternative actions, including those discussed elsewhere herein.

At block 202, in some embodiments, the server 40 may create a user account for the user of the client device 110 and relating to a well-being application executing thereon. The user account may include a user profile, linked accounts associated with the user, user preferences, or other information relevant to user well-being. For example, information regarding user health, finances, family, social connections, goals, or recommendation preferences may be provided from the user or determined based upon user-provided information. The user may further provide information regarding user social media accounts or grant access to the well-being application to access such information. The user account information may be updated over time as user conditions change.

At block 204, the server 40 may obtain user data relating to user well-being. The user data may include user interactions with the well-being application or other data from the well-being application executing on the client device 110. Such user interaction data may include data directly provided by the user via a user interface or data obtained by the well-being application monitoring user activity, which may include the well-being application accessing data generated by other applications of the client device 110. The user interaction data may include metadata regarding user selections, interactions with, or dismissal of prompts or other data presented via the well-being application.

Such user interactions may include whether a user views information relating to various topics, regardless of whether the user enters or updates data associated with such topics. For example, a user may view information relating to purchasing a new home, which may be stored as user interaction data relating to user property or finances. Similarly, user research within the well-being application of life insurance policies or adding beneficiaries may be stored as interaction data relating to user health, finances, or family.

The user data may further include user external data obtained by the server 40 (directly or via the well-being application) from additional data sources 41-45. Such user external data may include data from third party data sources, as well as data from separate data sources associated with the developer or provider of the well-being application. The user external data may include information regarding user accounts from an account data source 41, which may be financial, insurance, service, utility, health, or other types of accounts. For example, information regarding user gym membership accounts or insurance policies may be obtained from one or more account data sources 41.

Similarly, user external data relating to health of the user (or other users linked with the user in the well-being application, such as a spouse, child, or parent) may be obtained from one or more health data sources 42. User external data relating to social media posts from to social media feeds associated with the user (e.g., user posts, comments, views, or actions) may be obtained from one or more social data sources 43. For example, user posts and metadata may be collected for analysis to determine user connectedness or social or mental well-being. Financial data associated with the financial well-being of the user may likewise be obtained from one or more financial record data sources 44, such as bank records, investment records, insurance records, loan records, credit records, or other financial data associated with the user. Official record data sources 45 maintained by various governmental entities may be accessed to obtain information regarding user property ownership, residence, legal proceedings, or other information relevant to determining user conditions.

At block 206, the server 40 may determine one or more user conditions relating to user well-being based upon the user data. Such user conditions may be used to determine whether a triggering event has occurred or to determine well-being tasks following occurrence of a triggering event. The user conditions may include or be related to major life events, such as a birth, a death, an adoption, a marriage, a divorce, a change in employment, a retirement, a medical diagnosis, a purchase of real property, a change of residence, or reaching a threshold age (e.g., a retirement age or related age threshold, a health-related age threshold, or an age threshold associated with an insurance risk level change). Some user conditions may be determined directly based upon user external data entries obtained by the server 40 or user data entry via the user interface of the well-being application. Some user conditions may be determined indirectly by analysis of user external data or of user interactions with the well-being application.

In some embodiments, user conditions such as major life events may be determined by analysis of social media feeds associated with the user (e.g., user posts or posts referencing the user). User conditions such as current financial, mental, or social well-being states or levels may also be indirectly determined based upon analysis of social media feeds, which may include analysis of metadata (e.g., frequency or time of day trends of user posts).

At block 208, the server 40 may determine whether a triggering event has occurred based upon the one or more user conditions. The triggering event may be a predefined value or state of a user condition, or it may be a change of a user condition between states. In some embodiments, the identification of a new major life event for the user may be a triggering event. Some triggering events may be based upon time elapsed from a major life event, such as time since a birth of a user's child for a relevant time interval (e.g., six months, two years, five years, etc.). Thus, triggering events may be identified over the course of a relevant time interval based upon user conditions associated with past major life events.

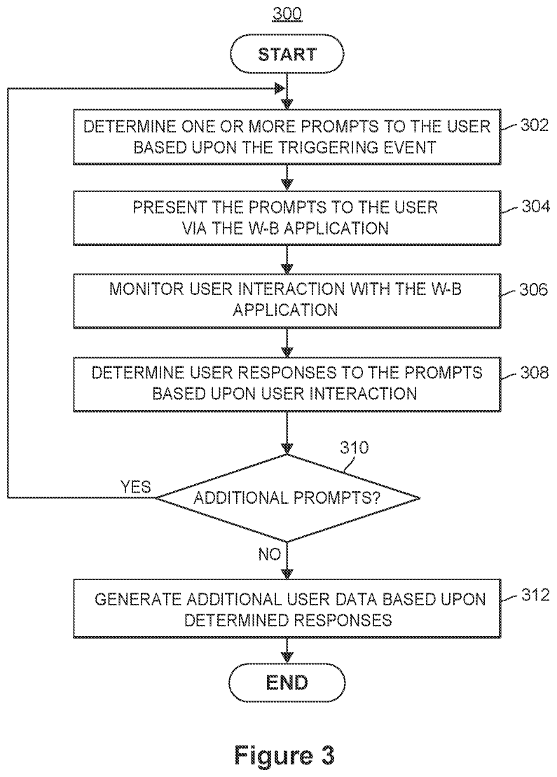

At block 210, in some embodiments, the server 40 may obtain additional user data upon identification of a triggering event. Such additional user data may be used to verify the triggering event or to specify details regarding a user condition. In some embodiments, such additional user data may be collected by monitoring user interaction with the well-being application, or by obtaining additional user external data. In further embodiments, obtaining the additional user data may include presenting one or more well-being prompts to the user, as discussed further below with respect to FIG. 3.

At block 212, the server 40 may determine one or more well-being tasks for the user based upon the determined user conditions, the user data, the additional user data, or a combination thereof. The well-being tasks indicate tasks for the users to perform relating to the financial, mental, social, or community well-being of the user. If additional user data has been obtained by presenting well-being prompts, the well-being tasks may be determined based at least in part upon the user response (or non-response) to such well-being prompts. The well-being tasks may include collecting financial documents (e.g., asset title documents, registrations, bank account information, or utility account information), uploading financial documents to a server associated with the well-being application (e.g., in the database 46 of the server 40 or in an account data source 41) or storing a list of financial information on such server, participating in a community volunteering activity (e.g., signing up to volunteer for a local volunteer event), connecting with an acquaintance (e.g., in person or via phone or social media), planning a vacation or other travel, making or reviewing retirement financial plans, performing property maintenance (e.g., seasonal preparation or periodic preventative or restorative maintenance at a residence), filing official form documents (e.g., tax returns, voter registration, or change of address or mail forwarding forms), updating insurance policy beneficiaries (e.g., to add or remove individuals following major life events), or providing information to insurance policy beneficiaries (e.g., for future planning purposes, such as for life insurance beneficiaries).

In some embodiments, the server 40 may determine a set of recommendations associated with well-being tasks. Such recommendations may include performing the tasks, preparing to perform the tasks, or learning more about the tasks. In some such embodiments, determining the set of recommendations may include applying an artificial intelligence (AI) model to the user data (including any additional user data) to determine one or more well-being tasks and generate one or more corresponding recommendations. This may include generating an input data set from the user data (i.e., any user interaction data, user external data, or additional user data), which may include selecting or preprocessing (e.g., reformatting or combining) a subset of such user data for analysis. One or more AI models may be selected based upon the triggering event, user conditions, or additional user data. The input data set may then be analyzed by applying the one or more AI models to the input data set to generate a plurality of rating scores associated with the respective well-being tasks that could be recommended to the user.

Such rating scores may be numeric quality ratings of the well-being tasks, probabilities of user action on the well-being tasks if presented to the user, or expected values of the well-being tasks (e.g., values derived based upon probabilities of user actions and estimated impact for the user if acted upon). Based upon such rating scores, one or more of the well-being tasks may be selected for inclusion in a recommendation to the user. For example, the highest-rated well-being task may be selected, or a group of tasks with the highest combined rating may be selected. The selected set of one or more well-being tasks may then be further tailored to the specific conditions or circumstances of the user.

At block 214, the server 40 may generate one or more user-specific well-being recommendations based upon the determined well-being tasks. In some embodiments, generating the user-specific well-being recommendations for the user may include personalizing aspects of the recommendation to increase the likelihood of the user performing the recommended well-being tasks. This may include selecting presentation options or timing for the recommendations, providing additional information regarding the well-being tasks, presenting the well-being tasks over time as a set of steps to be followed by the user based upon the user's level of sophistication or familiarity with such tasks, or otherwise adding specific details relevant to the user in performing the well-being task recommendations.

In some embodiments, generating a user-specific well-being recommendation may comprise identifying specific steps to perform the well-being task, identifying specific documents, or identifying specific individuals or organizations to contact. To generate such user-specific well-being recommendations, the server 40 may use the user data (including additional user data) to populate a general model of a well-being task, such as by identifying documents based upon known accounts and assets of the user or by identifying information not already available in the user data.

In some embodiments, user sentiment from social media feeds or other sources (e.g., user interactions with the well-being application) may be used to determine the best time or manner of presenting the recommendations. User sentiment metrics or other metrics relating to past user activities may further be used to determine recommendation details, such as recommending the user contact a particular person based upon past user interactions with such person.

At block 216, the server 40 may cause the one or more user-specific well-being recommendations to be presented to the user via the user interface of the well-being application executing on the client device 110. The user-specific well-being recommendations may be presented as pop-up or push notifications or alerts to the user via the user interface, may be sent via e-mail or other electronic messaging means, or may be presented as an audio recommendation via a speaker of the client device 110. In some embodiments, a user-specific well-being recommendation may be presented to the user by inclusion in a list or feed of information to the user by the well-being application. Such recommendations may be presented as offers to the user to learn more about a related topic, with user-specific well-being recommendations presented when the user selects or accepts such an offer. Additional or alternative presentation methods may be used in various embodiments.

FIG. 3 illustrates a flow diagram of an exemplary data collection method 300 using prompts to obtain additional user data. The computer-implemented method 300 may be performed in response to determining the occurrence of a triggering event in order to obtain further relevant details of a user condition to provide the user with well-being recommendations tailored to current user conditions. Various parts of the method 300 may be implemented by or performed using the various front-end components 2 and back-end components 4, which may communicate via the network 3, as described above. In some embodiments, a server 40 may perform the method 300 by collecting and processing user data obtained from a well-being application on a client device 110, and by causing the well-being application to present prompts to the user and receive responses from the user.

The exemplary data collection method 300 begins with determining one or more well-being prompts to present to the user in response to the occurrence of the triggering event (block 302). The one or more well-being prompts are then presented to the user via the user interface of the well-being application (block 304). User interaction with the well-being application is the monitored (block 306), and used to determine user responses to the one or more well-being prompts (block 308). Upon determination of such user responses, the need for additional well-being prompts may be determined (block 310). When no further well-being prompts are needed, additional user data is generated based upon the user responses (block 312). Such additional user data may then be used to generate user-specific well-being recommendations, as discussed above. The method 300 is exemplary only, and other methods may include additional, fewer, or alternative actions, including those discussed elsewhere herein.

At block 302, the server 40 may determine one or more well-being prompts to present to the user in order to obtain additional information. Such well-being prompts may be determined based upon the triggering event, determined user conditions, or other user data. In some instances, the well-being prompts may be selected to confirm or correct user data. In further instances, the well-being prompts may be determined to collect information that is identified as existing but missing in the user data (e.g., specific details of identified conditions, assets, connections, or goals).

In some embodiments, the well-being prompts may include direct questions to solicit specific detailed information or open-ended questions to prompt the user to provide information from which further questions may be determined and presented to the user. In further embodiments, the well-being prompts may include offers of additional information relating to a user condition or other well-being topics. Such offers of information may be selected to test user interest in a possible type of well-being task, without asking direct questions in an obtrusive manner. In this way, the method 300 may unobtrusively prompt the user for additional user information without significantly affecting the user experience of using the well-being application.

At block 304, the server 40 causes the well-being application of the client device 110 to present the determined one or more well-being prompts to the user to solicit user response. When a plurality of such well-being prompts are presented to the user, they may be presented either sequentially or concurrently. In some embodiments, presentation of some of such plurality of well-being prompts may be contingent upon the received or observed user responses to previous prompts. The well-being prompts may be presented as questions to the user, or may be presented as options or offers to the user to learn additional information about a relevant well-being-related topic.

In some embodiments, the well-being prompts may not be identified as such, rather being part of a set of information or options presented to the user. In this way, such well-being prompts may solicit user responses (e.g., taking actions or providing information within the well-being application) in an unobtrusive manner that does not significantly change the user experience with the well-being application. For example, well-being prompts offering the user additional information or asking whether the user would like additional information regarding a topic may be presented in a user feed or dashboard within the well-being application.

At block 306, the server 40 may monitor user interaction with the well-being application to determine user responses to the one or more well-being prompts. Since the user responses to such prompts may be either explicit (e.g., direct user answers to questions) or implicit (e.g., user acceptance of an offer to receive additional information about a well-being topic), the user interactions may be monitored over a time interval to obtain data or metadata from which to determine user responses. For example, an observed user interaction with the well-being application in response to a well-being prompt may be a direct response of providing additional information (e.g., by entering such data via a user interface). As another example, an observed user interaction with the well-being application in response to an information offer may include the user selecting an option to receive the additional information or, conversely, the user not selecting such an option after presentation during a time interval. In such example, the user selection may be taken as indicating user interest in the information (and related well-being topic), while user non-selection may be taken as an indication of user disinterest or irrelevance of the topic to the user.

At block 308, the server 40 may determine user responses to the one or more well-being prompts based upon observed user interaction with the well-being application. In some instances, user responses may be directly received from the user in response to direct questions. In further instances, the user responses may be inferred from user interactions to prompts that do not directly request answers to specific questions. In either case, the user interactions may be used to determine the user's responses to the one or more well-being prompts, and indications of such responses may be stored in the database 46 for further use in determining additional well-being prompts or in generating user-specific well-being recommendations.

At block 310, the server 40 may determine whether additional well-being prompts should be presented to the user based upon the determined user responses to the previous well-being prompts. If additional prompts are to be presented (e.g., to present direct questions after identifying user interest in a well-being topic or to explore an additional well-being topic), the server 40 determines the additional prompts at block 302 above. If no additional prompts are to be presented, the server 40 proceeds to generate additional user data based upon the determined user responses to the well-being prompts at block 312. Generating such additional user data may include formatting, processing, extracting, and/or storing user data entries from the determined responses. Such additional user data entries may be stored in the database 46 with the previously obtained user data for use in determining well-being tasks and generating user-specific well-being recommendations, as discussed above.

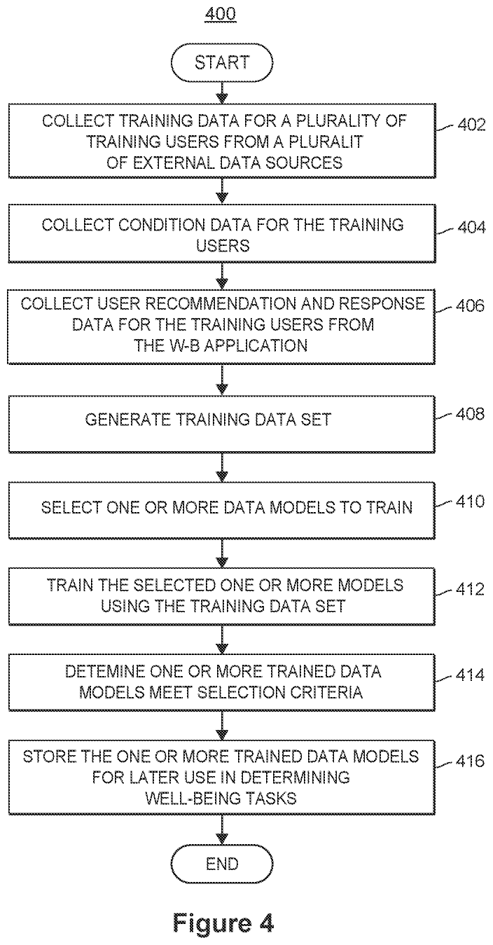

FIG. 4 illustrates a flow diagram of an exemplary model training method 400 for training an artificial intelligence (AI) model for determining well-being tasks. The model training method 400 may be implemented by one or more servers 40 of the well-being application system 100 to train a predictive AI model for determining well-being tasks for users based upon user data. The model training method 400 or another similar method may be implemented in advance of user interactions with the well-being application in order to obtain one or more models for predicting appropriate well-being tasks based upon user data, as discussed above. Such trained AI models may be stored in database 46 for later use.

The model training method 400 begins by collecting training data for a plurality of training users from a plurality of external data sources (block 402) and collecting condition data for the plurality of training users (block 404). Data regarding user recommendations that had been presented to the plurality of training users and the responses of the training users to such recommendations are further collected (block 406). The collected training user data and recommendation/response data are combined to generate a training data set (block 408). One or more data models are selected for training on the training data set (block 410) and are trained using the training data set (block 412), until one or more trained data models meet selection criteria (block 414). The one or more successfully trained data models are then stored as AI models for further use in determining well-being tasks to recommend to a user (block 416). In some embodiments, the exemplary method 400 may be modified to include additional, fewer, or alternative actions, including those discussed elsewhere herein.

At block 402, the server 40 may obtain a training data for a plurality of training users for training the AI model. The training data may include user external data obtained from a plurality of additional data sources 41-45. The training data associated with a plurality of training data users may be collected into a set of training data entries associated with the training data users. Such training data entries may include data regarding various aspects of user financial, mental, social, or community well-being obtained from the additional data sources 41-45. In some embodiments, the training data entries may include processed data generated by the server 40 from the collected data.

At block 404, the server 40 may obtain condition data for the training users in order to train the AI model. The condition data may be collected from one or more additional data sources, such as information provided by the user during an insurance underwriting process or entered by the user into a well-being application. The condition data of the training data users may be incomplete, in which case the training data set may be limited to a subset of the plurality of training data users for which condition data is available. In some embodiments, user health data may be used to infer condition data for some training users based upon prespecified rules.

At block 406, the server 40 may further obtain user recommendation data indicative of well-being recommendations presented to the training users, as well as observed responses of the training users after receiving such recommendations. Such user recommendation data may be collected via communication with the well-being application in order to train the AI model to determine the most effective well-being tasks to recommend to future users of the well-being application. Thus, the recommendation data may be indicative of training user responses to generic recommendations or test recommendations relating to well-being tasks, rather than user-specific well-being recommendations. Since user recommendation data may not be available for all training data users (or may be incomplete for some training data users), the training data set may be limited to those training users for whom sufficient user recommendation data is available. In some embodiments, the user recommendation data may be obtained by monitoring user responses to user-specific well-being recommendations in order to update an AI model previously used to determine well-being tasks for such user-specific well-being recommendations.

At block 408, the server 40 may then merge the training data from the external data sources with the condition data and the user recommendation data to generate a training data set. As indicated above, such training data set may be limited to such training users for whom all three types of data are available. The training data set may comprise a plurality of training data entries connecting recommendations and responses of training data users with data regarding their well-being and condition prior to (and potentially after) receiving well-being recommendations.

At block 410, the server 40 may select one or more untrained data models to train using the training data set. The selected data models may include any type of untrained machine learning models for supervised or unsupervised learning. A model may be specified based upon user input specifying relevant parameters to use as predicted variables (e.g., indications of user interest, responses to recommendations, or user well-being changes after receiving well-being recommendations) and other variables to use as potential explanatory variables (e.g., user conditions, user well-being metrics, or other user characteristics of the training users). For example, a model may be specified to predict the likelihood of a user performing a recommended well-being task based upon the collected training user data. Conditions for training the AI model may likewise be selected, such as limits on model complexity or limits on model refinement past a certain point. Because outcomes may vary significantly by certain user characteristics such as age, the models may also be selected to specify certain user characteristics, and multiple models may be trained for different groups of training users. In some embodiments, unsupervised machine learning techniques may be used to determine the relevant user characteristics based upon the training data set.

At block 412, the server 40 may train the selected one or more untrained data models using the training data set. To train the data models, the server 40 may randomly select a first subset of the training data entries to use in generating a trained data model. The selected data model may then be trained on the first subset of training data entries using appropriate machine learning techniques, based upon the type of model selected and any conditions specified for training the model.

The model may be trained using a supervised or unsupervised machine-learning program or algorithm. The machine-learning program or algorithm may employ a neural network, which may be a convolutional neural network, a deep learning neural network, or a combined learning module or program that learns in two or more features or feature datasets in particular areas of interest. The machine-learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine-learning algorithms and/or techniques.

Machine-learning may involve identifying and recognizing patterns in existing data in order to facilitate making predictions for subsequent data. In some embodiments, due to the processing power requirements of training machine learning models, the selected model may be trained using additional computing resources (e.g., cloud computing resources) based upon data provided by the server 40. Such training may continue until at least one model is validated and meets selection criteria to be used as a predictive model.

At block 414, the server 40 may determine that one or more trained data models meet selection criteria to be selected as an AI model for determining well-being tasks for user-specific well-being recommendations. Thus, each trained data model may be validated using a second subset of the training data records to determine model accuracy and robustness. Such validation may include applying the trained model to the training data records of the second subset of training data records to predict values usable for selecting well-being tasks to recommend to users (e.g., probabilities of users performing the well-being tasks). The trained model may then be evaluated to determine whether the model performance is sufficient based upon the validation stage predicted values. The sufficiency criteria applied may vary depending upon the size of the training data set available for training, the performance of previous iterations of trained models, or user-specified performance requirements. When the server 40 determines the trained model has not achieved sufficient performance, additional training may be performed at block 412, which may include refinement of the trained model or retraining on a different first subset of the training data records, after which the new trained model may again be validated and assessed at block 414. When the server 40 determines the trained model has achieved sufficient performance at block 414, the trained model may be stored for later use.

At block 416, the server 40 may store the one or more selected trained data models for later use in determining well-being tasks to recommend to users in user-specific well-being recommendations according to the methods and techniques disclosed herein. The trained AI models may be stored as sets of parameter values or weights for analysis of further user data, which may also include analysis logic or indications of model validity in some instances. Thus, a plurality of models may be stored for determining well-being tasks to recommend under different sets of input data conditions. In some embodiments, trained predictive models may be stored in the database 46 associated with server 40.

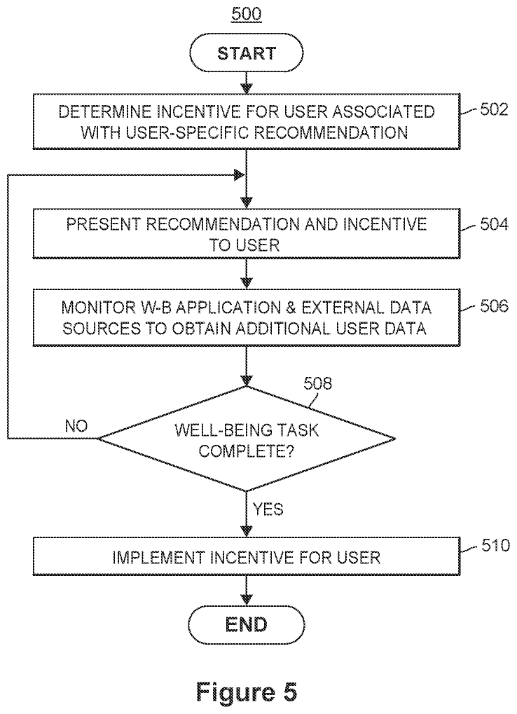

FIG. 5 illustrates a flow diagram of an exemplary incentive selection and implementation method 500 for incentives associated with user performance of well-being tasks. The computer-implemented method 500 may be performed to provide incentives to users for performing the recommended well-being tasks presented according to the methods described above. Various parts of the method 500 may be implemented by or performed using the various front-end components 2 and back-end components 4, which may communicate via the network 3, as described above. In some embodiments, a server 40 may perform the method 500 by determining user incentives, by collecting and processing user data obtained from the well-being application on a client device 110 to monitor user performance of recommended well-being tasks, and by causing an incentive to be implemented when the user has met the incentive criteria by performing a recommended well-being task.

The exemplary incentive selection and implementation method 500 begins with determining one or more incentives for a user to encourage performance of well-being tasks to be included in a user-specific well-being recommendation (block 502). The user-specific well-being recommendation and an indication of the incentives are then presented to the user via the user interface of the well-being application (block 504). User interaction with the well-being application is the monitored (block 506) and used to determine whether a well-being task associated with an incentive has been completed by the user (block 508). Upon determination of such user completion of a well-being tasks associated with an incentive, the incentive is then implemented to reward the user for performing the recommended well-being task (block 510). The method 500 is exemplary only, and other methods may include additional, fewer, or alternative actions, including those discussed elsewhere herein.

At block 502, the server 40 may determine one or more incentives associated with one or more well-being tasks included in the user-specific well-being recommendation. Incentives may include benefits, bonuses, or discounts for completing well-being tasks, such as discounts on insurance policies, discounts on well-being services (e.g., well-being application subscriptions, gym memberships, or health food delivery services). In some embodiments, portions of an incentive may be associated with completion of different well-being tasks, such as well-being tasks that form a multi-step process or well-being tasks that must all be completed to obtain the incentive. Some incentives may include a specified time interval within which the corresponding well-being tasks must be completed to meet the criteria of the incentive. The incentives may be determined as part of determining the well-being tasks at block 212, as part of generating the user-specific well-being recommendation at block 214, or as a separate action between or following such actions.

At block 504, the server 40 may cause a user-specific well-being recommendation and corresponding incentives to be presented to the user via the user interface of the client device 110, as described in block 216 above. In some embodiments, the one or more incentives may be included in the user-specific recommendation presented to the user. In further embodiments, the one or more incentives may be presented to the user separately or subsequently to the presentation of the corresponding user-specific well-being recommendation. For example, information regarding the incentive may be presented to the user upon an indication of user interest in the corresponding user-specific well-being recommendation. In still further embodiments, an indication of an incentive may be presented to the user prior to information regarding one or more well-being tasks associated with the incentive (i.e., prior to the user-specific well-being recommendation) in order to entice the user to consider the recommended well-being tasks.

At block 506, after presentation of the incentive information and corresponding well-being tasks to the user, the server 40 may monitor the user data from the well-being application and additional data sources 41-45 to obtain additional user data regarding the recommended well-being tasks associated with the one or more incentives. In some embodiments, this may include both collecting additional user data from the well-being application regarding user interaction with the application (e.g., user activities or data entered by the user), and collecting additional user data from one or more additional data sources 41-45 associated with one or more well-being tasks to be performed by the user to meet the incentive criteria.

Such monitoring may continue only as long as the incentive remains in effect, which may be a limited time interval or may be a periodically renewing time interval. For example, an incentive may be ongoing and require user performance of a well-being task on a periodic basis (e.g., reviewing financial plans on an annual basis to ensure they remain up to date).

At block 508, the server 40 may determine whether one or more well-being tasks associated with an incentive are complete based upon the additional user data. If the well-being tasks are not complete, the server 40 may periodically cause a reminder to be presented to the user, such as by presenting an indication of the incentive and relevant portions of the corresponding user-specific well-being recommendation to the user via the user interface of the well-being application on the client device 110 at block 504, followed by continued monitoring at block 506.

If the well-being tasks associated with an incentive have not been completed within a required time interval, additional or alternative incentives may in some cases be determined and presented to the user at block 504. In other cases, the method 500 may simply terminate without implementing an incentive upon expiration of the time interval. If the well-being tasks are determined to have been completed such that the user meets the criteria for an incentive, the method 500 may proceed to implement such incentive at block 510.

At block 510, the server 40 causes the incentive to be implemented for the user based upon the determined completion of the one or more well-being tasks required to meet the criteria for the incentive. Implementing the incentive may include communicating an indication that the user meets the criteria for the incentive to a server associated with an incentive service, which may be associated with an additional data source 41-45 or other external system via the network 3. In some embodiments, this may include communicating a discount to a server associated with a user account (e.g., associated with an account data source 41), which may be a user account with an insurer or other financial organization, a subscription service, a retailer, or other provider of goods or services to the user. Arrangements regarding implementation of user incentive discounts or other benefits may be prearranged between the provider of the well-being application and the provider of the incentive benefit. After causing implementation of the incentive benefit, the method 500 ends.

### OTHER MATTERS

Although the preceding text sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning.

With the foregoing, an insurance customer may opt in to a program to receive a reward, insurance discount, or other type of benefit. In some aspects, customers may opt in to a rewards, loyalty, or other program associated with use of the well-being application, such as a rewards program that collects data and provides incentives for performing well-being tasks. The customers may therefore allow a remote server to collect sensor, telematics, biometric, mobile device, and other types of data discussed herein. With customer permission or affirmative consent, the data collected may be analyzed to provide certain benefits to customers. For instance, insurance cost savings may be provided to customers based upon reducing their risk through improving their well-being. Recommendations that lower risk or provide cost savings to customers may also be generated and provided to customers based upon data analysis, as discussed elsewhere herein. Other functionality or benefits of the systems and methods discussed herein may also be provided to customers in return for them allowing collection and analysis of the types of data discussed herein. In return for providing access to data, risk-averse insured customers may receive discounts or insurance cost savings on home, renters, vehicle, personal articles, life, health, and other types of insurance from the insurance provider.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more modules of a computer system (e.g., a processor or a group of processors) may be configured by software (e.g., an application or application portion) as a module that operates to perform certain operations as described herein.

In various embodiments, a module may be implemented mechanically or electronically. For example, a module may comprise dedicated circuitry or logic that is permanently configured (e.g., as a special-purpose processor, such as a field programmable gate array (FPGA) or an application-specific integrated circuit (ASIC) to perform certain operations. A module may also comprise programmable logic or circuitry (e.g., as encompassed within a general-purpose processor or other programmable processor) that is temporarily configured by software to perform certain operations. It will be appreciated that the decision to implement a module mechanically, in dedicated and permanently configured circuitry, or in temporarily configured circuitry (e.g., configured by software) may be driven by cost and time considerations.

Accordingly, the term “module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which modules are temporarily configured (e.g., programmed), each of the modules need not be configured or instantiated at any one instance in time. For example, where the modules comprise a general-purpose processor configured using software, the general-purpose processor may be configured as respective different modules at different times. Software may accordingly configure a processor, for example, to constitute a particular module at one instance of time and to constitute a different module at a different instance of time.

Modules can provide information to, and receive information from, other modules. Accordingly, the described modules may be regarded as being communicatively coupled. Where multiple such modules exist contemporaneously, communications may be achieved through signal transmission (e.g., over appropriate circuits and buses) that connect the modules. In embodiments in which multiple modules are configured or instantiated at different times, communications between such modules may be achieved, for example, through the storage and retrieval of information in memory structures to which the multiple modules have access. For example, one module may perform an operation and store the output of that operation in a memory device to which it is communicatively coupled. A further module may then, at a later time, access the memory device to retrieve and process the stored output. Modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented modules. The performance of certain of the operations may be distributed among the one or more processors, not only residing within a single machine, but deployed across a number of machines. In some example embodiments, the one or more processors or processor-implemented modules may be located in a single geographic location (e.g., at a location of a mobile computing device or at a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information. Such memories may be or may include non-transitory, tangible computer-readable media configured to store computer-readable instructions that may be executed by one or more processors of one or more computer systems.

As used herein any reference to “one embodiment” or “an embodiment” means that a particular element, feature, structure, or characteristic described in connection with the embodiment is included in at least one embodiment. The appearances of the phrases “in one embodiment,” “in an embodiment,” “in some embodiments,” or similar phrases in various places in the specification are not necessarily all referring to the same embodiment or the same set of embodiments.

Some embodiments may be described using the terms “coupled,” “connected,” “communicatively connected,” or “communicatively coupled,” along with their derivatives. These terms may refer to a direct physical connection or to an indirect (physical or communicative) connection. For example, some embodiments may be described using the term “coupled” to indicate that two or more elements are in direct physical or electrical contact. The term “coupled,” however, may also mean that two or more elements are not in direct contact with each other, but yet still co-operate or interact with each other. Unless expressly stated or required by the context of their use, the embodiments are not limited to direct connection.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless the context clearly indicates otherwise.

This detailed description is to be construed as exemplary only and does not describe every possible embodiment, as describing every possible embodiment would be impractical, if not impossible. One could implement numerous alternate embodiments, using either current technology or technology developed after the filing date of this application.

Upon reading this disclosure, those of skill in the art will appreciate still additional alternative structural and functional designs for the systems and methods disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

Finally, the patent claims at the end of this patent application are not intended to be construed under 35 U.S.C. § 112(f), unless traditional means-plus-function language is expressly recited, such as “means for” or “step for” language being explicitly recited in the claims. The systems and methods described herein are directed to an improvement to computer functionality, which may include improving the functioning of conventional computers in performing tasks.

What is claimed is:

1. A computer-implemented method for generating user-specific well-being recommendations, comprising:
monitoring, by one or more processors, user interactions with a well-being application via a user interface of a client computing device associated with the user;
collecting, by the one or more processors, user external data from one or more external data sources;
identifying, by the one or more processors, occurrence of a triggering event associated with a user condition based upon the user interactions and the user external data;
determining, by the one or more processors, a well-being recommendation to the user to complete a well-being task based upon the triggering event and at least one of the user interactions or the user external data, the well-being task being a task related to user financial, mental, or social well-being;
generating, by the one or more processors, a user-specific recommendation to perform the well-being task; and
causing, by the one or more processors, the user-specific recommendation to be presented to the user via the user interface of the client computing device.

2. The computer-implemented method of claim 1, further comprising:
determining, by the one or more processors, one or more well-being prompts to the user based upon the triggering event;
causing, by the one or more processors, the one or more well-being prompts to be presented to the user via the user interface of the client computing device; and
receiving, at the one or more processors, an indication of a user response to at least one of the one or more well-being prompts,
wherein the well-being recommendation is determined in part based upon the indication of the user response.

3. The computer-implemented method of claim 2, wherein:
the at least one of the one or more well-being prompts comprises an offer of additional information relating to the user condition; and
the user response comprises accepting the offer of additional information.

4. The computer-implemented method of claim 1, wherein:
the one or more external data sources include a social media feed associated with the user; and
identifying the triggering event comprises identifying a major life event of the user based upon social media data included in the social media feed.

5. The computer-implemented method of claim 1, wherein the user condition comprises at least one of the following major life events associated with the user or with another person associated with the user: a birth, a death, an adoption, a marriage, a divorce, a change in employment, a retirement, a medical diagnosis, a purchase of real property, a change of residence, or reaching a threshold age.

6. The computer-implemented method of claim 1, wherein the well-being task comprises at least one of the following: collecting financial documents, uploading financial documents to a server associated with the well-being application, participating in a community volunteering activity, connecting with an acquaintance, planning a vacation, reviewing retirement financial plans, performing property maintenance, filing official form documents, updating insurance policy beneficiaries, or providing information to insurance policy beneficiaries.

7. The computer-implemented method of claim 1, wherein generating the user-specific recommendation comprises identifying specific steps to perform the well-being task, identifying specific documents, or identifying specific individuals or organizations to contact.

8. The computer-implemented method of claim 1, wherein determining the user-specific recommendation comprises:
generating, by the one or more processors, an input data set from the user interactions and the user external data relating to the triggering event;
applying, by the one or more processors, an artificial intelligence model associated with the triggering event to the input data set to generate a plurality of rating scores associated with respective well-being tasks; and
selecting, by the one or more processors, one or more of the well-being tasks to include in the well-being recommendation based upon the rating scores.

9. The computer-implemented method of claim 1, wherein the user-specific recommendation includes an incentive for performing the well-being task within a specified time interval.

10. The computer-implemented method of claim 9, further comprising:
collecting, by the one or more processors, additional data associated with the user following presentation of the user-specific recommendation during the specified time interval;
determining, by the one or more processors, completion of the well-being task by the user; and
causing, by the one or more processors, the incentive to be implemented for the user based upon the completion of the well-being task.

11. A computer system for generating user-specific well-being recommendations, comprising:
one or more processors;
a non-transitory memory communicatively coupled to the one or more processors and storing executable instructions that, when executed by the one or more processors, cause the computer system to:
monitor user interactions with a well-being application via a user interface of a client computing device associated with the user;
collect user external data from one or more external data sources;
identify occurrence of a triggering event associated with a user condition based upon the user interactions and the user external data;
determine a well-being recommendation to the user to complete a well-being task based upon the triggering event and at least one of the user interactions or the user external data, the well-being task being a task related to user financial, mental, or social well-being;
generate a user-specific recommendation to perform the well-being task; and
cause the user-specific recommendation to be presented to the user via the user interface of the client computing device.

12. The computer system of claim 11, wherein:
the executable instructions further cause the computer system to:
determine one or more well-being prompts to the user based upon the triggering event;
cause the one or more well-being prompts to be presented to the user via the user interface of the client computing device; and
receive an indication of a user response to at least one of the one or more well-being prompts; and
the well-being recommendation is determined in part based upon the indication of the user response.

13. The computer system of claim 12, wherein:
the at least one of the one or more well-being prompts comprises an offer of additional information relating to the user condition; and
the user response comprises accepting the offer of additional information.

14. The computer system of claim 11, wherein the executable instructions that cause the computer system to generate the user-specific recommendation further cause the computer system to identify specific steps to perform the well-being task, identify specific documents, or identify specific individuals or organizations to contact.

15. The computer system of claim 11, wherein:
the user-specific recommendation includes an incentive for performing the well-being task within a specified time interval; and
the executable instructions further cause the computer system to:
collect additional data associated with the user following presentation of the user-specific recommendation during the specified time interval;
determine completion of the well-being task by the user; and
cause the incentive to be implemented for the user based upon the completion of the well-being task.

16. A tangible, non-transitory computer-readable medium storing executable instructions for generating user-specific well-being recommendations that, when executed by one or more processors of a computer system, cause the computer system to:
monitor user interactions with a well-being application via a user interface of a client computing device associated with the user;
collect user external data from one or more external data sources;
identify occurrence of a triggering event associated with a user condition based upon the user interactions and the user external data;
determine a well-being recommendation to the user to complete a well-being task based upon the triggering event and at least one of the user interactions or the user external data, the well-being task being a task related to user financial, mental, or social well-being;
generate a user-specific recommendation to perform the well-being task; and
cause the user-specific recommendation to be presented to the user via the user interface of the client computing device.

17. The tangible, non-transitory computer-readable medium of claim 16, wherein:
the executable instructions further cause the computer system to:
determine one or more well-being prompts to the user based upon the triggering event;
cause the one or more well-being prompts to be presented to the user via the user interface of the client computing device; and
receive an indication of a user response to at least one of the one or more well-being prompts; and
the well-being recommendation is determined in part based upon the indication of the user response.

18. The tangible, non-transitory computer-readable medium of claim 17, wherein:
the at least one of the one or more well-being prompts comprises an offer of additional information relating to the user condition; and
the user response comprises accepting the offer of additional information.

19. The tangible, non-transitory computer-readable medium of claim 16, wherein the executable instructions that cause the computer system to generate the user-specific recommendation further cause the computer system to identify specific steps to perform the well-being task, identify specific documents, or identify specific individuals or organizations to contact.

20. The tangible, non-transitory computer-readable medium of claim 16, wherein:
the user-specific recommendation includes an incentive for performing the well-being task within a specified time interval; and
the executable instructions further cause the computer system to:
collect additional data associated with the user following presentation of the user-specific recommendation during the specified time interval;
determine completion of the well-being task by the user; and
cause the incentive to be implemented for the user based upon the completion of the well-being task.
